(19) United States
(12) Patent Application Publication
(10) Pub. No.: US 2022/0054049 A1
(43) Pub. Date: Feb. 24, 2022

(54) HIGH-PRECISION TEMPORAL MEASUREMENT OF VIBRO-ACOUSTIC EVENTS IN SYNCHRONISATION WITH A SOUND SIGNAL ON A TOUCH-SCREEN DEVICE

(71) Applicants: UNIVERSITE DE MONTPELLIER, Montpellier (FR); NATURALPAD, PRADES-LE-LEZ (FR)

(72) Inventors: Simone Dalla Bella, Montréal (CA); Sébastien Andary, Saint-Gely Du Fesc (FR)

(21) Appl. No.: 17/415,173

(22) PCT Filed: Dec. 23, 2019
(86) PCT No.: PCT/EP2019/086899

§ 371 Date: Jun. 17, 2021

(30) Foreign Application Priority Data
Dec. 21, 2018 (FR) 1873999

(51) Int. Cl. A61B 5/12; A61B 5/00; H04R 1/08; G10L 25/51

(52) U.S. Cl. CPC A61B 5/123; G10L 25/51; H04R 1/08; A61B 5/748

(57) ABSTRACT

A method for determining the tap time of a user in response to an auditory stimulus. The method includes: playing back the auditory stimulus corresponding to a reference audio signal, recording the reference audio signal and one or more vibro-acoustic events, the vibro-acoustic events following one or more taps by the user on a touch screen of the device in reaction to the reference audio signal, detecting the reference audio signal in the recorded audio signal, the recorded audio signal is recorded by a microphone of the touch-screen device, during the step of detecting the reference audio signal in the recorded audio signal, placing the reference audio signal and the recorded audio signal on a common time scale, filtering the detection and normalizing it to keep only the frequencies corresponding to the vibro-acoustic events generated by the user's actions on the touch screen, and detecting the vibro-acoustic events in which instants associated with the vibro-acoustic events are identified in the signal obtained after filtering.

### TECHNICAL FIELD OF THE INVENTION

The invention relates to a method for determining the tap times of a user in response to an auditory stimulus. For the implementation of the method according to the invention, a touchscreen device is available, which will be better described in the following.

In the scope of the invention, the user is a person capable of perceiving at least one sound signal. References to certain parts of the body of the user, in particular his fingers or his hands, are intended only to provide a better understanding of the interactions that the user is likely to have with the touchscreen device used to implement the method of the invention.

The method according to the invention finds its application in particular in the study of the auditory-motor synchronization. The method according to the invention could be implemented in the context of experimental studies seeking to analyze the auditory-motor coordination in the user, or simply could be implemented in educational games aimed at improving the auditory-motor coordination in children for example. These are non-limiting examples of applications of the present invention.

### BACKGROUND

We know the methods of visual-motor coordination whose aim is to study the coordination of the movements of certain segments of the body of a person, with the visual information perceived simultaneously by said person. Recently, such methods have been deployed on touchscreen devices to measure the reaction time of a user in response to visual stimuli emitted from a touchscreen device.

Reference may be made to the document US 2014/0249447 which describes a method based on the recording of acoustic vibrations generated by a user when tapping on a touchscreen device in response to visual stimuli, constituting targets for the user.

The principle of the method is as follows. A series of instructions is initialized by means of a program installed on a tablet-type device. The program asks the tablet to start the recording of the acoustic vibrations. This recording being stored in an audio file on an appropriate support of the tablet as well as the chronology of said recording. The user can then start a trial by tapping on the center of a touchscreen of the device. Then the program asks the tablet to light up one of the targets displayed on the screen and to write the time at which the target lit up in the logbook. The goal is that the user hit the target that has lit up. At the same time, the microphone of the tablet allows to pick up the vibrations created by the contact of the user with the target and the time at which the vibrations are detected by said microphone is indicated in the logbook. The tablet then receives a contact notification from the touchscreen and the precise coordinates of the location of the contact and writes the time at which the notification was received and the coordinates of the area, on the touchscreen, where the contact occurred.

In the described method, the synchronization of the visual stimuli with the taps of the user is made possible by storing the chronology of the events from acoustic vibrations and the target lighting in the logbook file system of the tablet. The tap time is not determined at the end of this synchronization. This synchronization method is not applicable to a method involving auditory stimuli since in this case it is not possible to store the exact chronology of the auditory stimulus played by loudspeakers in the logbook files, and even less so for a sound emitted by a emitter device external to the tablet.

Also known are the methods in which processing is applied to an audio signal recorded by the microphone of a touchscreen device or by sensors placed in proximity to the touchscreen of the device. In most cases, the goal is to suppress unwanted vibrations generated by the taps of a user on the touchscreen device or other events such as an unintentional contact with the device, without extracting information from the peaks of the signal associated with these vibrations.

The document EP 2 875 419 discloses such a method. The principle of the method is as follows. For one or more sensors, an audio signal emitted from a microphone is processed to determine one or more portions of the signal associated with a touch of the touchscreen. A joint estimation of a plurality of the determined parts is performed to isolate a signal characteristic of a typing of the user on the touchscreen. The identified portion may include a desired signal component, such as a recorded human voice, and an undesired signal component, such as the sound associated with the typing of the user on the touchscreen. The signal characteristic of a typing on the touchscreen determined in the previous step is removed from the corresponding audio signal for each of the received audio signals. A signal combiner can then be used to match the temporal location of the signal characteristic of the typing with the corresponding portion in the received signal when subtracted from said signal. The resulting audio signals are then summed and output.

The methods proposed so far do not address the high-accuracy temporal measurement of the tap times of a user in synchronization with a sound signal on a touchscreen device.

### SUMMARY OF THE INVENTION

The invention provides a method for determining tap times of a user in response to an auditory stimulus, the method comprising the following steps:
playing back the auditory stimulus, said auditory stimulus corresponding to the reference audio signal,
recording the reference audio signal and one or more vibro-acoustic events, said vibro-acoustic events following one or more taps by the user on a touchscreen of a touchscreen device in reaction to the reference audio signal, the signal resulting from the recording being referred to as the recorded audio signal,
detecting the reference audio signal in the recorded audio signal.

The method is characterized in that:
the recorded audio signal is recorded by means of a microphone of the touchscreen device,
during the step of detecting the reference audio signal in the recorded audio signal, the reference audio signal and the recorded audio signal are placed on a single common time scale,
it comprises a filtering step in which the recorded signal obtained subsequently to the detecting step is initially filtered and then normalized in such a way to keep only the frequencies corresponding to the vibro-acoustic events generated by the actions of the user on the touchscreen, and in that
it comprises a step of detecting vibro-acoustic events in which instants associated with said vibro-acoustic events are identified in the signal obtained at the end of the filtering step.

The method according to the invention thus allows on the one hand to identify the beginning of the reference audio signal in the recorded audio signal and from then to determine the instants associated with the taps of the user on the touchscreen.

According to various characteristics of the invention which may be taken together or separately:
the step of detecting the reference audio signal in the recorded audio signal comprises a first sub-step of normalizing the recorded audio signal so as to obtain a normalized recorded audio signal;
the step of detecting the reference audio signal in the recorded audio signal comprises a second sub-step during which the reference audio signal is resampled at the same rate as the recorded audio signal and normalized so as to obtain a resampled and normalized reference audio signal;
the normalization operation performed during the step of detecting the reference audio signal in the recorded audio signal is performed by means of a normalization function;
the step of detecting the reference audio signal in the recorded audio signal comprises, subsequent to the first and the second sub-steps, a third sub-step of identifying the beginning of the resampled and normalized reference audio signal in the normalized recorded audio signal;
the third sub-step comprises a sub-step of constructing a suitable filter so as to identify in the normalized recorded audio signal the most probable sampling instant that corresponds to the beginning of the K first samples of the resampled and normalized reference audio signal,
the third sub-step comprises a sub-step of determining a time window of size in which searching for the normalized resampled reference audio signal in the normalized recorded audio signal,
the third sub-step comprises a sub-step for determining the sampling instant;
the filtering step comprises a first filtering sub-step and optionally a second filtering sub-step;
the first filtering sub-step is implemented by means of a 1st order Butterworth type bandpass filter having a low frequency of 50 Hz and a high frequency of 200 Hz, and
the second filtering sub-step is implemented by means of a 1st order Butterworth type low-pass filter having a high frequency of 400 Hz, so that at the end of the second filtering sub-step a filtered normalized recorded audio signal is obtained;
subsequent to the second filtering sub-step, the filtering step comprises a third sub-step of local normalization of the filtered normalized recorded audio signal obtained at the end of the second filtering sub-step so as to obtain a filtered normalized recorded audio signal, said local normalization sub-step being carried out by means of a local normalization function;
subsequent to the filtering step, the step of detecting vibro-acoustic events is implemented;
the step of detecting vibro-acoustic events comprises a first sub-step of determining the energy of the filtered normalized recorded audio signal, said first sub-step being performed by means of an energy function;
the step of detecting vibro-acoustic events comprises, subsequent to the first sub-step, a second sub-step of smoothing the signal obtained at the end of the first sub-step by means of a smoothing function defined by the convolution product of the signal with a Hamming-type weighting window;
the step of detecting vibro-acoustic events comprises, subsequent to the second sub-step, a third sub-step of extracting, from the smoothed signal, the set of P sampling instants corresponding to local maxima and/or onset candidates of vibro-acoustic events;
the step of detecting vibro-acoustic events comprises, subsequent to the third sub-step, a fourth sub-step of pre-selecting the onset candidates of vibro-acoustic events;
the fourth sub-step comprises a sub-step of grouping candidates associated with the P sampling instants according to a first selection criterion, said first selection criterion corresponding to the grouping of the candidate sampling instants spaced by a predetermined number m of samples, so as to form groups of local maxima;
the fourth sub-step comprises a sub-step of conserving in each group the instants associated with the local maxima for which the maximum value of the energy is obtained;
the step of detecting vibro-acoustic events comprises, subsequent to the fourth sub-step, a fifth sub-step of removing the spurious maxima;
the fifth sub-step comprises a sub-step of sorting, by decreasing height, the local maxima kept at the end of the fourth sub-step;
the fifth sub-step comprises a sub-step of conserving the ρNₜₐₚ largest local maxima, Nₜₐₚ being the number of vibro-acoustic events comprised in the measurement time window and ρ being strictly greater than 1, ρ>1;
the method further comprises, subsequent to the step of detecting the vibro-acoustic events, an additional step of optimizing the signal obtained at the end of said step of detecting the vibro-acoustic events,
said optimizing step comprises a sub-step of pairing the set of instants tᵢ, with i<ρNₜₐₚ, of the local maxima kept in step of conserving the largest ρNₜₐₚ local maxima with the set of model instants tⱼ, with j<Nₜₐₚ, measured by the touchscreen;
said optimizing step comprises a sub-step of evaluating the quality of the pairing performed during the sub-step S600) by means of an objective function,
said optimizing step comprises a sub-step of maximizing the objective function by means of a parameter,
said optimizing step comprises a sub-step of selecting the local maxima associated with the sampling instants which are exactly paired to the model instants measured by the touchscreen;
the optimizing step comprises, subsequent to the fourth sub-step, a fifth adjusting sub-step during which the local maxima selected at the end of the fourth sub-step are adjusted so as to conform to the recorded audio signal;
the adjustment is performed by applying a phase shift compensation function to the maxima.

The invention also relates to a touchscreen device suitable for implementing the method as described above. The device comprises a touchscreen, a microphone, a central processing unit configured to perform at least steps of the method as described above.

The invention further relates to a computer program comprising instructions which when the program is executed by the computer cause the computer to implement the method as previously described.

The invention also relates to a computer storage medium on which the computer program as described above is stored.

### BRIEF DESCRIPTION OF FIGURES

Further objects, characteristics and advantages of the invention will become clearer in the following description, made with reference to the attached figures, in which:

FIG. 1 schematically illustrates the process of acquiring the signal from the taps of the user,

FIG. 2 schematically illustrates the processing of the recording and the extraction of the instants associated with the taps of the user and the time reference,

FIG. 3 illustrates steps of the method of determining the tap times of the user in response to an auditory stimulus,

FIG. 4 illustrates the sub-steps of the step of detecting the reference audio signal in the recorded audio signal, the secondary sub-steps are illustrated in dotted lines,

FIG. 5 illustrates the sub-steps of the step of filtering the recorded signal obtained subsequent to the third step and normalization,

FIG. 6 illustrates the sub-steps of the step of detecting the vibro-acoustic events in the signal obtained at the end of the fourth step,

FIG. 7 illustrates the sub-steps of the step of detecting the vibro-acoustic events in the signal obtained at the end of the fourth step (continued),

FIG. 8 illustrates the sub-steps of the step of optimizing the signal obtained at the end of the fifth step.

### DETAILED DESCRIPTION OF THE INVENTION

### A. General Comments and Terminology

The terms defined in this section are applicable to all embodiments disclosed in the following description.

In the scope of the invention, a touchscreen device comprises at least a touchscreen, an audio system comprising at least a microphone and a loudspeaker, an audio playback and recording program, a computer program adapted to implement the method according to the invention.

The touchscreen device further comprises at least one processing unit which can be defined by a Central Processing Unit (CPU), said CPU being adapted to execute the audio playback and recording program and the computer program adapted to implement the method according to the invention.

According to the architecture, the touchscreen device further comprises at least one Graphics Processing Unit (GPU), dedicated to parallelizable matrix computing, having a Random Access Memory (RAM) and a storage medium, for example a Random Access Memory (RAM), an Electrically-Erasable Programmable Read-Only Memory (EEPROM) or a Flash memory. The audio playback and recording program is recorded on said storage medium. Similarly, the program adapted to implement the method according to the invention is recorded on the storage medium.

The method can also be implemented by means of an application-specific integrated circuit (ASIC) processor or a field-programmable gate array (FPGA), or directly implemented by means of a microprocessor optimized to run digital signal processing (DSP) applications. These types of processors thus allow to improve the processing performance in the touchscreen device.

Preferably, the touchscreen device is a mobile device such as a touch tablet or a smartphone. More generally, the touchscreen device may be a computer with a touchscreen.

The structure of the touchscreen device or frame serves as a support for all other elements of the device, namely the touchscreen, the audio system, the processor.

The touchscreen is an interactive screen allowing data to be acquired during events affecting its surface. The surface is more precisely an interactive tile or interaction surface, preferably flat and rectangular in shape.

In the scope of the invention, the interaction surface is suited to undergo instantaneous deformations, i.e. adapted to vibrate and dissipate the vibratory energy created upon contacts with said interaction surface. The contact or the contacts may result either from an interaction between the fingers of the user and the interaction surface or from an interaction between a stylus provided therefor and said interaction surface or from any other object with the interaction surface. In any case, the contact necessarily originates from an action of the user. In the scope of the invention, this action is a typing, a tap or a touch by the user.

The vibratory energy emitted by the interaction surface during the taps is adapted to propagate, from close to close, through the elements of the touchscreen device in the form of a wave by inducing an elastic deformation of the various elements of the device through which the wave passes, causing acoustic waves. A “vibratory path” is created between the interaction surface and the elements of the device through which said wave passes. In particular, the vibrational energy dissipated by the interaction surface is, at a minima, adapted to propagate from said interaction surface to the microphone, through the structure of the device or any other element of the device, and adapted to interfere with said microphone. In the scope of the invention, such a phenomenon will be referred to as a vibro-acoustic signal. In addition, the contact with the interacting surface at the origin of the vibro-acoustic signal will be referred to as a vibro-acoustic event, noted eᵥₐ.

The touchscreen may further comprise a detection system. The detection system defines the type of touchscreen used. It is composed of a plurality of detection elements allowing to identify the coordinates of the areas where contacts with the touchscreen occur. There are different detection modes in the touchscreens. For the former, a direct contact with the touchscreen is required to trigger the detection, while for the latter, touching the touchscreen is not required for the detection to occur.

The detection elements are typically sensors, detectors or emitters. They can be located below the interaction surface or in corners on either side of the screen. These detection elements allow to provide a measure of the number of typing made by the user through the touchscreen, which is an input data within the scope of the invention. In any event, the touchscreen used to implement the invention is not limited to any particular detection mode.

The loudspeaker may be any apparatus able to produce or emit a sound or sounds from an electrical signal or electrical signals. In other words, the loudspeaker is any apparatus able to convert an electrical signal into a sound or sounds. These sounds may, in particular, come from a music or from an audio pre-recorded in the memory of the device and whose playback by the loudspeaker or the loudspeakers is adapted to be triggered by the user through the audio playback and recording program. They are detectable by the microphone of the device. Moreover, the application of the invention to the study of the auditory-motor synchronization requires, in the case where a loudspeaker is used, that these sounds can be perceptible by the user of the touchscreen device. However, it is not mandatory to use the loudspeaker or the loudspeakers.

The sound or the sounds can be emitted by a metronome. Similar to a sound or sounds that would be emitted by the loudspeaker, the application of the invention to the auditory-motor synchronization requires that the sound emitted by such an instrument be at least perceptible by the user of the touchscreen device. The metronome can be used in combination with the loudspeaker or the loudspeakers. Other devices known to the person skilled in the art for emitting sounds, for example musical instruments, can be used alone or in combination depending on the application. The invention is not limited to the above examples.

The sounds emitted by the loudspeaker or the loudspeakers and/or the metronome and/or the sound-emitting device are called auditory stimuli. In the scope of the invention, the auditory stimuli generated by the sound-emitting device are referred to as the reference audio signal or reference signal, denoted S_{R}. In response to these auditory stimuli, and depending on their duration, the user is likely to have one or more reactions, in this case, he or she is in particular led to touch the interaction surface of the touchscreen, and to cause one or more vibro-acoustic events, eᵥₐ.

The sounds emitted by the loudspeaker or the loudspeakers and/or the metronome can be preceded and/or interrupted by a silence or silences. Silence means the absence of sound. In other words, the silence corresponds to an interruption of sound.

The microphone may be any apparatus allow to detect an acoustic and/or vibro-acoustic signal or signals and adapted to convert said signal or signals into electrical signal or signals. The microphone is able to record an acoustic signal or signals. It allows to provide an electrical voltage proportional to the acoustic variations. The acoustic signal may originate from a vibro-acoustic event or vibro-acoustic events on the interaction surface, eᵥₐ, a sound or sounds from noises in the immediate environment of the device, e.g. the sound emitted by a human voice, the emission of sounds by the loudspeaker or the loudspeakers of the device. The acoustic signal can still come from a metronome as mentioned above. The acoustic signal may be damaged by noise from the equipment itself.

The acoustic signals of interest refer in particular to the sounds emitted by the sound-emitting device (possibly preceded and/or interspersed by silences) and those resulting from the tap of the user (vibro-acoustic events, also possibly preceded and/or interspersed by silences).

The vibro-acoustic signals, Sᵥₐ, resulting from the events eᵥₐ are located in a particularly low frequency range, well below the sound waves. The accuracy of detection and recording of such signals by the microphone is only limited by the capabilities of the microphone itself. Typically, for the devices such as touch tablets and smartphones, the microphones used have a sampling frequency of between 8 and 48 kHz. These values are not limiting in the scope of the present invention. Although an optimum detection accuracy is preferable, it is not necessary for the implementation of the method according to the invention.

The detection accuracy is in the broad sense the fidelity of reproduction by the microphone of the sound or the sounds emitted by the device, the loudspeakers and/or the metronome and/or the sound-emitting device. The detection accuracy should not be confused with the quality of the recording or the recorded signal. The quality of the recorded audio signal depends, in addition to the detection accuracy, on the nature, the shape, etc. of the object hitting the interaction surface, but also on the speed with which the object hits the surface. For example, the mechanical vibrations and/or sounds emitted by a stylus will be perceived more distinctly than the mechanical vibrations and/or sounds emitted by a finger of the user when the user is wearing gloves, regardless of the touchscreen detection mode.

In the following sections, we focus on the processing of the signal and/or the signals. The reference signal S_{R} and the mechanical vibrations from the taps of the user are recorded by the microphone. The reference audio signal S_{R} and the mechanical vibrations may interact and create interference. This interference results in a complex resultant signal formed from the superposition of single vibrations. These single vibrations may have different amplitudes and frequencies and are determined so that the resulting signal may undergo frequency and/or amplitude variations over time. The resulting signal can be of any form. It can be formed from a mixture of sinusoidal signals. This resulting signal is transmitted to the microphone and then recorded by the latter.

The signal transmitted and recorded by the microphone is called recorded audio signal or recorded signal, denoted S_{E}. As mentioned above, the acoustic and/or vibro-acoustic signal of signals detected by the microphone is or are converted into an electrical signal or signals, which in turn is or are converted and stored as digital data at the sampling rate of the touchscreen device used, in the memory of said device. In this regard, the operation during which the resulting signal is sampled or measured at regular and defined intervals, in particular according to the sampling rate of the microphone, is referred to here as sampling. The microphone may also preferably be associated with a conversion device in the touchscreen device.

In the scope of the invention, it is sought to synchronize the start of the reference signal S_{R} and the vibro-acoustic events eᵥₐ in order to determine the number of taps by the user. However, the emission start instant of the reference signal S_{R} relative to the start instant of the vibro-acoustic events eᵥₐ is unknown since the clock of the processor itself is unknown. In this respect, the reference signal S_{R} and the recorded signal S_{E} are therefore susceptible to undergo successive mathematical transformations, also called signal processing operations of the signal or processing and described in the following.

The resampling operation consists in bringing a first signal and a second signal to the same sampling frequency and amplitude. For this purpose, one of said first and second signals is normalized, for example the first signal. The other signal, i.e. the second signal, undergoes a resampling at the same frequency, i.e. at the same sampling rate, as the first signal and then normalized as well. In other words, the second signal is “re-recorded” in a different sampling format than it was originally. For example, the first signal may be the recorded signal S_{E} while the second signal may be the reference signal S_{R}.

The normalization is understood here as the operation of modifying and/or correcting the amplitude of a parameter signal in such a way that, over a measurement time window, the maximum value of the amplitude of said parameter signal reaches a predetermined value, and that the correction made for this maximum value of the amplitude is also applied to the entire parameter signal, i.e. over the entire measurement time window. For example, the parameter signal can be the recorded signal S_{E}.

According to a first computing mode, a normalization function can be defined as follows:

$${f}_{n 1} ( X ) := \frac{X - mean ( X )}{median ( abs ( X ) )} \qquad ( 1 )$$

Where X is the parameter signal,
mean (X) is the mean value of the signal X,
abs (X) is the signal whose the samples are absolute values of the samples or elementary signals of the signal X,
median (abs(X)) is the median value of the abs(X) signal.

The mean value, mean (X), where RMS value is the mean amplitude of the parameter signal over a given time window. In other words, it is the mean amplitude of all the samples or elementary signals that make up the parameter signal X.

The median value, median (X), corresponds to the amplitude allowing the amplitudes of all the samples or elementary signals composing the parameter signal X to be cut into two parts each comprising the same number of samples so that a first half of the samples has amplitudes greater than the median value and the other half of the samples has amplitudes less than said median value.

The computing of the normalization function is carried out in the measurement window, i.e. over the entire recording time, also called session. In other words, the normalization function is applied to each of the samples or elementary signals composing the parameter signal X. This being the case, the time window used may have a more limited size so that the computing of the normalization function is performed in a limited time window, the so-called weighting window.

In either case, the computing of the normalization function is performed in a “fixed” time window, meaning that the mean is computed over the entire time window considered, i.e. the measurement window or the weighting window. In other words, the mean is computed for all the samples within the time window considered.

The computing of the normalization function can also be performed in a time window, the so-called sliding window. The sliding time window allows a weighted moving mean transformation of a quantity associated with the parameter signal X for each subset of the sliding window and for a preselected number of samples. In one computing mode of the normalization function over a sliding window, said normalization function is recomputed for each subset of the sliding window to obtain the normalized values for the entirety of said sliding window. This allows a smoothing of the values by removing the transient fluctuations that may appear in the signal. This is called a local normalization function.

According to a second computing mode, the local normalization function can be defined as follows:

$${f}_{nloc} ( X ) := \frac{X - {mean}_{loc} ( X )}{{mean}_{loc} ( {abs}_{loc} ( X - {mean}_{loc} ( X ) ) )} \qquad ( 2 )$$

Where X is the parameter signal,
mean_{loc} (X) is the local mean value of the signal X,
abs_{loc} (X−mean_{loc} (X)) is the signal whose the samples are local
absolute values of the samples or elementary signals of the signal X over the subset of the sliding window.

The notions of “measurement window”, “weighting window”, “fixed window” and “sliding window” can be extended to the various processing of the signal and mathematical transformations applied to the parameter signal of the present invention.

In addition, one or more filtering operations may also be applied to the signal. The filtering operation can have several objectives depending on the processing to be performed.

A suitable filter can be constructed and used to locate in a first parameter signal, noted X₁, the most probable sampling instant that corresponds to the beginning of the K first samples of a second parameter signal, noted X₂. The suitable filter provides an amplification of the samples of the second parameter signal X₂ with respect to the first parameter signal X₁, and allows to identify the location of these samples with more or less precision depending on the method chosen to build said filter. In other words, the suitable filter allows to temporally detect and maximize one or more samples in said first parameter signal.

A time window of size T₂ can be defined to restrict the search for the second parameter signal X₂ in the first parameter signal X₁.

In an embodiment of the invention, the sampling instant t₂ corresponding to the start of the second signal X₂ in the first signal X₁ is defined as follows:

$${t}_{2} := {argmax}_{t < {T}_{2}} {\sum }_{k = t}^{t + K} {X}_{1} ( k ) \cdot {X}_{2} ( k - t ) \qquad ( 3 )$$

Where t corresponds to a given sampling instant,
t₂ expresses the sampling instant corresponding to the start of the second parameter signal X₂ in the first parameter signal X₁,
T₂ is the size of the time window (in seconds).

The maximum argument, noted argmax, designates the set of the samples, over the time window T₂, in which the convolution product Σₖ₌ₜ^{t+K}S₁(k)·S₂(k−t) reaches its maximum value.

More precisely, we are looking for the sample that maximizes the convolution product of the first parameter signal X₁ by the second parameter signal X₂ reversed in time. The sampling instant associated with this sample will constitute the reference time used for the synchronization of said first and second parameter signals X₁, X₂.

The operation of synchronizing a first parameter signal X₁ with a second parameter signal X₂ consists in positioning said signals on a single common time scale. This synchronization operation of the signals therefore requires the ability to accurately detect the start of the second parameter signal X₂ in the first parameter signal X₁. The sampling instant associated with the start of the second parameter signal X₂ is then considered as the origin instant of a time reference. In other words, it consists in locating the sampling time that corresponds to the beginning of the reference audio signal S_{R}. Thus, the moment of the taps and the moment of the beginning of the reference signal S_{R} are positioned on a single common time scale.

Other methods can be considered by the person skilled in the art to build the suitable filter by taking into account the sampling frequency and the size of the time window.

In addition, a bandpass filter can be used to keep in a parameter signal X only the frequencies associated with useful events, for example the vibro-acoustic events eᵥₐ, while the frequencies associated with any other event, for example a sound signal, noises from the external environment or noises coming from the equipment itself, are attenuated. This attenuation is possible because only the events with frequencies located in a frequency range between a first limit frequency, called low frequency, and a second limit frequency, called high frequency, of the filter are kept.

In one embodiment of the invention, the bandpass filter used is a Butterworth filter. The Butterworth filter is a linear filter with a relatively constant response in the bandpass.

The invention is not limited to the use of Butterworth bandpass filters. Other bandpass filters known to the person skilled in the art and having a relatively constant response in the bandpass can be envisaged, without going beyond the scope of the invention. An example is the Bessel filter, which also offers a very flat response in the bandpass.

Furthermore, in addition to any normalization and filtering operations, one or more smoothing operations may be applied to the parameter signal X. The smoothing operation allows to attenuate peaks, known as secondary peaks, which would consist of disturbances or noise not associated with useful events, for example vibro-acoustic events eᵥₐ, and to keep only the peaks, known as major peaks, of said parameter signal X.

By peak we mean values of the parameter signal X corresponding to local maxima. In principle, a secondary peak has a smaller amplitude than a major peak.

In one embodiment of the invention, the smoothing function is defined by the convolution product of the parameter signal X with a Hamming-type weighting window as follows:

\tilde{X}=X*Hamming(T_{smoothing}) (4)

Where \tilde{X} is the smoothed parameter signal,
X is the parameter signal before smoothing,
Hamming is the weighting window,
T_{smoothing} is the size of the weighting window.

The weighting window, Hamming, is a weighted moving mean transformation operation. It allows to smooth the values of the parameter signal X by applying a larger smoothed observation coefficient at the center of the window and smaller and smaller weights as one moves away from the center of the window.

The implementation of the above mentioned operations is made possible by means of a dedicated program installed on the touchscreen device. The processor allows to execute the instructions received by this program and the programs installed on said touchscreen device.

### B. Detailed Description of the Invention

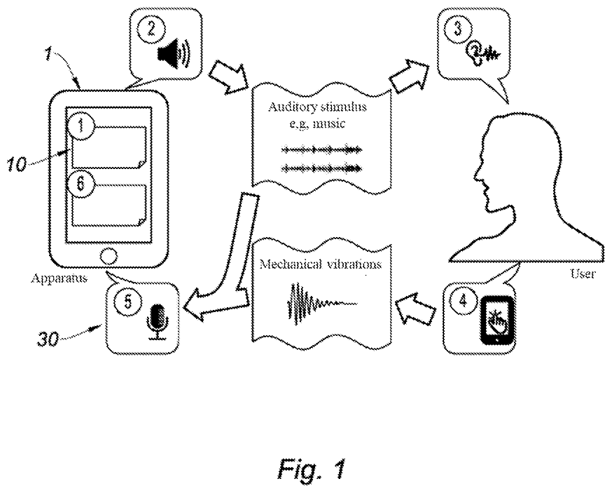

With reference to FIG. 1, the acquisition process of the signal is described. The audio playback and/or recording program starts the playback of the auditory stimulus on the touchscreen device 1.

The auditory stimulus or reference audio signal S_{R} is emitted from the loudspeakers of the touchscreen device. The user is then adapted to perceive the reference audio signal S_{R}, which causes the user to touch the touchscreen 10 of the device and generate one or more vibro-acoustic events eᵥₐ in synchronization with the reference audio signal S_{R}.

The microphone 30 of the touchscreen device then records both the reference audio signal S_{R} and the mechanical vibrations generated by the vibro-acoustic events generated by the taps of the user. The program then stores the resulting signal, recorded audio signal S_{E}, in the memory of the device 1. The memory of the device may be of the Random Access Memory (RAM), Electrically-Erasable Programmable Read-Only Memory (EEPROM) or Flash memory type.

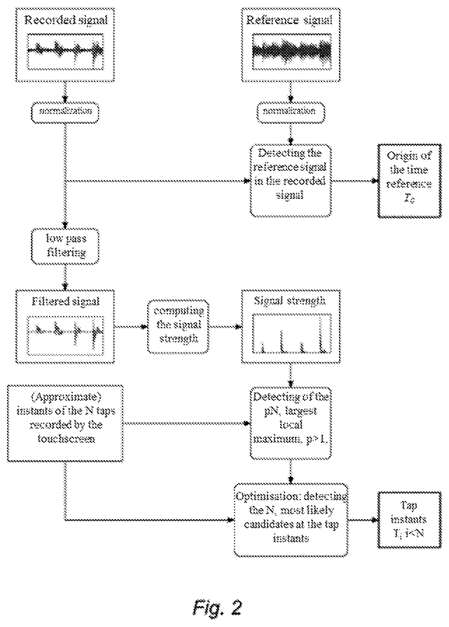

With reference to FIG. 2, the algorithm for processing the recorded audio signal S_{E} and extracting the instants associated with the taps of the user and the time reference is illustrated. This illustration helps to better understand the structure of the computing algorithm.

The inputs of the algorithm are the recorded audio signal S_{E}, the reference audio signal S_{R}, and the number of taps measured by the touchscreen 10 of the device. The outputs of the algorithm are the location of the beginning of the reference audio signal S_{R} in the recorded audio signal S_{E} and the instants of the taps of the user.

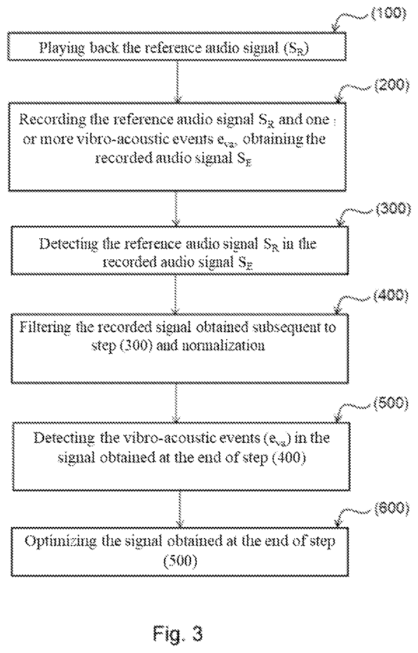

As illustrated in FIG. 3, the method of determining the tap times of the user according to the invention comprises the following steps:
100) playing back of a reference audio signal S_{R},
200) recording the reference audio signal S_{R} and one or more vibro-acoustic events eᵥₐ, said recording corresponding to the recorded audio signal S_{E},
300) detecting the reference audio signal S_{R} in the recorded audio signal S_{E},
400) filtering the recorded signal obtained during step 300),
500) detecting the vibro-acoustic events eᵥₐ in the signal obtained at the end of step 400), and
600) optimizing the signal obtained at the end of step 500).

Steps 100) to 600) are implemented in the above-mentioned order by means of a computer program. Several steps among steps 100) to 600) comprise sub-steps. These sub-steps and the order in which they are performed are better described in the following. In general, these sub-steps are presented in the order in which they are performed.

The first step 100) of playing back the reference audio signal S_{R} may be performed by means of a loudspeaker of the device 1 and/or a metronome and/or a sound-emitting device, depending on the intended application. Preferably, the loudspeakers and/or the metronome are preferred for playing back the reference audio signal S_{R}.

The second recording step 200) is performed by means of the microphone 30 of the device 1. The recording performed during this step is the recorded audio signal S_{E}.

The recorded audio signal S_{E} comprises a first main component derived from the reference audio signal S_{R} and a second main component originating from the vibro-acoustic events eᵥₐ. These components are not immediately differentiable and form a complex recorded audio signal.

As mentioned above, said vibro-acoustic events eᵥₐ are generated by the taps of the user on the touchscreen 10 of the device 1 in reaction to the reference audio signal S_{R}. These events therefore occur concomitantly with the playback of the reference audio signal S_{R}. Several processing operations of the recorded audio signal S_{E} are necessary to be able to determine the instants and times of these events eᵥₐ in said reference audio signal S_{R}. Indeed, it is important to note that the detection instants tⱼ measured by the touchscreen are not sufficient because their accuracy is between 50 and 300 milliseconds for commercially available touchscreen devices.

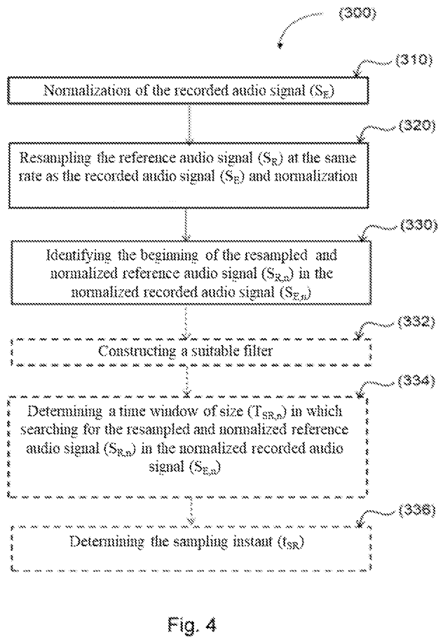

With reference to FIG. 4, the third step 300) of detecting the reference audio signal S_{R} in the recorded audio signal S_{E} is aimed at determining the origin of the time reference.

In this regard, step 300) may comprise a first sub-step 310) of normalizing the recorded audio signal S_{E}, after which a normalized recorded audio signal S_{E,n} is obtained.

Step 300) may further comprise a second sub-step 320) that may be implemented concomitantly with the first sub-step 310). The second sub-step 320) consist in resampling the reference audio signal S_{R} at the same rate as the recorded audio signal S_{E} and normalizing the resampled reference signal. At the end of this second sub-step 320), a resampled and normalized reference audio signal S_{R,n} is obtained.

Advantageously, the normalization operations respectively of said recorded audio signal S_{E} and said resampled reference audio signal are performed by means of a normalization function defined by:

$${f}_{n} ( X ) := \frac{X - mean ( X )}{median ( abs ( X ) )} \qquad ( 5 )$$

Where X here is the recorded audio signal (S_{E}) or the resampled reference audio signal (S_{R}),
   mean (X) is the mean value of the considered signal,
   abs (X) is the signal whose the samples are absolute values of the samples of the signal X and
   median (abs(X)) is the median value of the abs (X) signal.

The implementation of the first and second sub-steps 310) and 320) allows to obtain the normalized resampled reference signal S_{R,n} having the same sampling frequency and the same amplitude as the normalized recorded signal S_{E,n}. These sub-steps allow also to place the normalized resampled reference signal S_{R,n} and the normalized recorded signal S_{E,n} on a single time scale.

Preferably, step 300) may also comprises, subsequent to the first sub-step 310) and to the second sub-step 320), a third sub-step 330) of identifying the beginning of the normalized resampled reference audio signal S_{R,n} in the normalized recorded audio signal S_{E,n}. The third sub-step 330) comprises three secondary sub-steps identified in FIG. 4 by dotted boxes.

A first secondary sub-step 332) consist in constructing a suitable filter so as to identify in the normalized recorded audio signal S_{E,n} the most probable sampling instant t_{SR} that corresponds to the beginning of the K first samples of the resampled and normalized reference audio signal S_{R,n}. The suitable filter provides an amplification of the samples of the resampled and normalized reference audio signal S_{R,n} with respect to the normalized recorded audio signal S_{E,n}, and allows to identify the localization of these samples with more or less accuracy depending on the method chosen to build said filter.

The third sub-step 330) further comprises a second secondary sub-step 334) of determining a time window of size T_{SR,n} in which to search for the normalized resampled reference audio signal S_{R,n} in the normalized recorded audio signal S_{E,n}. The size T_{SR,n} of the time window corresponds to the number of samples comprised in the time window. The use of a time window of size T_{SR,n} allows to restrict the search for the beginning of the resampled and normalized reference audio signal S_{R,n} in the normalized recorded audio signal S_{E,n}.

The third sub-step 330) comprises a third secondary sub-step 336) of determining the sampling instant t_{SR}. In particular, this step aims to determine the origin of the time reference. The determination of the origin of the time reference also allows to determine from which instant the vibro-acoustic eventsₑᵥₐ are likely to occur.

The sampling instant t_{SR} is defined as follows:

$${t}_{{S}_{R}} := {argmax}_{t < {T}_{SR , n}} {\sum }_{k = t}^{t + K} {S}_{E , n} ( k ) \cdot {S}_{R , n} ( k - t ) \qquad ( 6 )$$

Where t corresponds to a given sampling instant,
t_{SR} expresses the sampling instant corresponding to the beginning of the resampled and normalized reference audio signal S_{R,n} in the normalized recorded audio signal S_{E,n},
T_{SR,n} is the size of the time window in seconds, and
argmax, denotes the set of the points, over the time window T_{SR,n}, at which the convolution product Σₖ₌ₜ^{t+H}S_{E,n} (k)·S_{R,n}(k−t) reaches its maximum value.

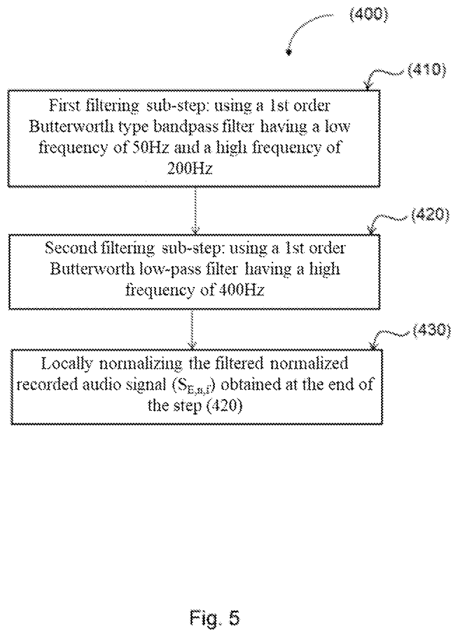

With reference to FIG. 5, the fourth step 400) of filtering the normalized recorded audio signal S_{E,n} obtained in step 300) aims at keeping in the normalized recorded audio signal only the frequencies associated with the vibro-acoustic events eᵥₐ. At the same time, the frequencies associated with the normalized resampled reference signal S_{R,n} and those associated with the possible noises coming from the external environment or the noises coming from the equipment itself, are attenuated.

In this regard, step 400) may comprise a first sub-step 410) of filtering the normalized recorded audio signal S_{E,n}. Preferably, it is implemented by means of a 1st order Butterworth type bandpass filter having a low frequency of 50 Hz and a high frequency of 200 Hz. That said, the sub-step 410) may be implemented by means of any other bandpass filter known to the person skilled in the art allowing to attenuate the unwanted frequencies of the normalized recorded audio signal S_{E,n} while having a relatively constant response within the bandpass.

Step 400) may optionally comprises a second sub-step 420) of filtering the signal obtained at the end of the first filtering sub-step 410). It is implemented by means of a 1st order Butterworth type low-pass filter with a high frequency of 400 Hz. It further allows to attenuate the frequencies associated with the normalized resampled reference signal S_{R,n}, and generally all the components of the normalized recorded audio signal S_{E,n} that are not associated with the taps of the user on the touchscreen 10. A Bessel filter that also provides a very flat response in the bandpass can also be used.

At the end of the first sub-step 410) and, if applicable, of the second sub-step 420), a filtered normalized recorded audio signal S_{E,n,f} is obtained.

Step 400) also comprises a third sub-step 430) of locally normalizing the filtered normalized recorded audio signal S_{E,n,f} obtained at the end of step 420). Preferably, the third sub-step 430) is implemented subsequent to the second sub-step 420).

The local normalization operation 430) is performed by means of a local normalization function defined by:

$${f}_{nloc} ( {S}_{E , n , f} ) := \frac{{S}_{E , n , f} - {mean}_{loc} ( {S}_{E , n . f} )}{{mean}_{loc} ( {abs}_{loc} ( {S}_{E , n , f} - {mean}_{loc} ( {S}_{E , n , f} ) ) )} \qquad ( 7 )$$

Where S_{E,n,f} is the filtered normalized recorded audio signal,
mean_{loc} (S_{E,n,f}) is the local mean value of the filtered normalized recorded audio signal S_{E,n,f},
abs_{loc} (S_{E,n,f}−mean_{loc} (S_{E,n,f})) is the signal whose the samples are local absolute values of the samples of the filtered normalized recorded audio signal S_{E,n,f} over the subset of a sliding window of size 2Tₙₒᵣₘ.

At the end of the third sub-step 430), a filtered normalized recorded audio signal [symbol] is obtained, to which a local normalization processing has been applied.

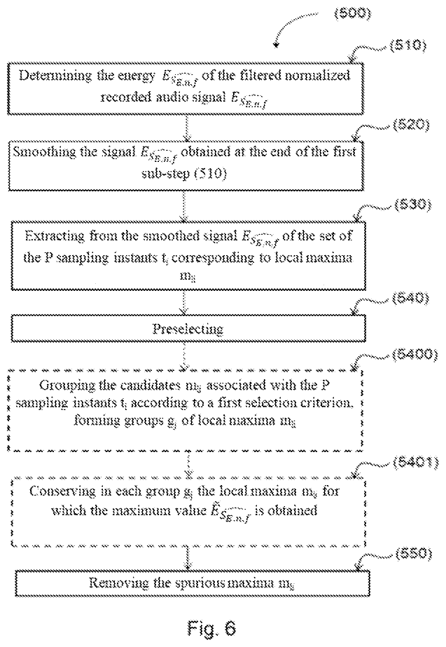

With reference to FIG. 6, the fifth step 500) of detecting the vibro-acoustic events eᵥₐ in the filtered normalized recorded audio signal [symbol] aims at identifying in said filtered normalized recorded audio signal [symbol] the instants of the peaks associated with the vibro-acoustic events eᵥₐ generated by the taps of the user. By “peaks” we mean the samples with maximum amplitudes over a given time window.

In this respect, step 500) advantageously comprises a first sub-step 510) of determining the energy [symbol] of the filtered normalized recorded audio signal [symbol] . Preferably, the first sub-step 510) is implemented by means of an energy function defined by:

$$( t ) = {\sum }_{k = - {T}_{E}}^{{T}_{E}} {\vert ( t + k ) \vert}^{2} \qquad ( 8 )$$

Where t corresponds to a given sampling instant,
2T_{E} is the size of a sliding time window, and
[symbol] is the filtered normalized recorded audio signal obtained at the end of step 430).

At the end of the first sub-step 510) the energy is obtained over the measurement time window for all the samples so that a “rough” identification of the instants associated with the vibro-acoustic events eᵥₐ is already possible. However, this signal may still comprises a certain number of peaks, so-called secondary peaks, which do not correspond to said vibro-acoustic event eᵥₐ.

In this regard, step 500) may comprises, subsequent to the first sub-step 510), a second sub-step 520) of smoothing the signal [symbol] . It is implemented by means of a smoothing function defined by the convolution product of the signal [symbol] with a Hamming-type weighting window:

[symbol]

:= [symbol] *Hamming(T_{smoothing}) (9)

Where [symbol] is the smoothed signal,
[symbol] is the signal before smoothing, Hamming is the weighting window, and
T_{smoothing} is the size of the weighting window.

This smoothing sub-step allows to keep only the major peaks in the signal [symbol] . Indeed, the use of the Hamming type weighting window allows to smooth the values of the signal [symbol] by applying a larger observation coefficient at the center of the window and weights that are weaker and weaker as one moves away from the center of the window. Thus, the secondary peaks are cancelled, while the peaks with the highest amplitude are kept.

At the end of the second sub-step 520) the signal corresponding to the energy is smoothed.

Advantageously, step 500) may comprise, subsequent to the second sub-step 520), a third sub-step 530) of extracting from the smoothed signal [symbol] the set of the P sampling instants tᵢ corresponding to local maxima mₗᵢ also called onset candidates of vibro-acoustic events eᵥₐ.

The said sampling instants corresponding to the beginning of extracted vibro-acoustic events eᵥₐ are such that:

[symbol]

(tᵢ)> [symbol] (tᵢ₋₁) (10)

[symbol]

(tᵢ)> [symbol] (tᵢ₊₁) (11)

As further illustrated in FIG. 6, step 500) comprises, subsequent to the third sub-step 530), a fourth sub-step 540) of pre-selecting the onset candidates of vibro-acoustic event eᵥₐ This fourth sub-step 540) is intended to remove the “bundles” of peaks that are too close together in time.

It is implemented by means of the secondary sub-steps 5400) and 5401) identified in FIG. 6 by dotted boxes.

A first secondary sub-step 5400) consists in performing a grouping of candidates mₗᵢ associated with the P sampling instants tᵢ according to a first selection criterion. The first selection criterion corresponds to the grouping of candidate sampling instants spaced by a predetermined number m of samples. The candidate instants meeting the first selection criterion are then grouped into groups gⱼ,

A second secondary sub-step 5401) consists in keeping in each group gᵢ the instants associated with the local maxima mₗᵢ for which the maximum value of [symbol] is obtained. In this way, for each group gᵢ of samples only one sampling instant tᵢ is kept.

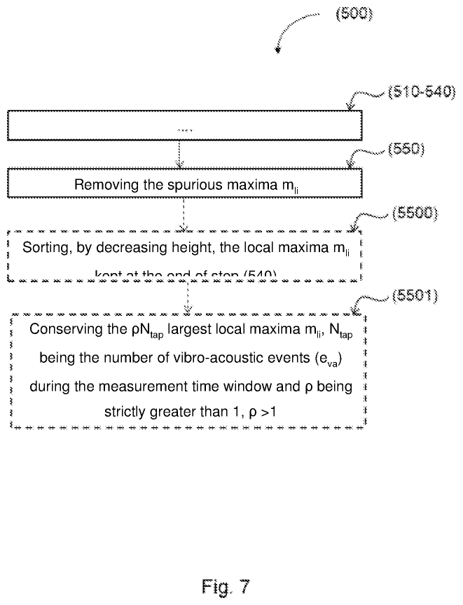

As illustrated in FIG. 7, step 500) may comprises, subsequent to the fourth sub-step 540), a fifth sub-step 550) of removing the spurious maxima mₗᵢ. Indeed, even if a preselection is carried out during step 540), the signal may still contain spurious peaks which it is preferable to remove.

Spurious peaks can be, for example, false positives. “False positives” are peaks that are considered to be local maxima mₗᵢ when they should not be. The fifth sub-step 550) is not mandatory, in particular if the signal does not contain spurious peaks. In practice, however, it is highly likely that such peaks are present in the signal.

The fifth sub-step 550) comprises secondary sub-steps 5500) and 5501) identified in the figure by dotted boxes.

The fifth sub-step comprises a first sub-step 5500) of sorting, by decreasing height, the local maxima mₗᵢ kept at the end of step 540). It should be understood here that, independently of the groups gᵢ to which the maxima mₗᵢ belong, said maxima mₗᵢ are sorted by decreasing height.

Then, during a second secondary sub-step 5501), ρNₜₐₚ larger local maxima mₗᵢ, where Nₜₐₚ is the number of vibro-acoustic events eᵥₐ comprised in the measurement time window and ρ being strictly greater than 1 (ρ being a natural number, ρ>1) are kept with respect to the sorting, by decreasing height, performed during step 5500).

The fifth sub-step 550) allows for a more refined identification of the instants associated with the vibro-acoustic events eᵥₐ in the signal [symbol] . The tap times of the user can be computed with a relative accuracy.

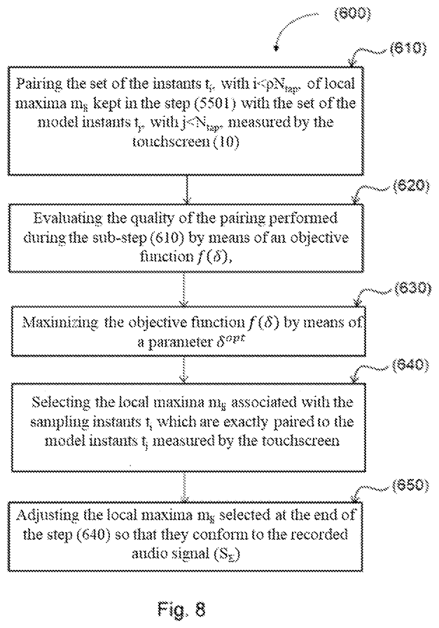

With reference to FIG. 8, the method according to the invention further comprises a step of optimizing the signal resulting from step 500). The optimization step aims at precisely identifying the maxima mₗᵢ corresponding to the vibro-acoustic events eᵥₐ by a tracing or a superimposing of the signal resulting from step 500) with the measurement of the taps of the user performed by the touchscreen 10.

Step 600) comprises a first sub-step 610) of pairing the set of instants tᵢ, with i<ρNₜₐₚ, of the local maxima mₗᵢ kept in step 5501) with the set of model instants tⱼ, with j<Nₜₐₚ, measured by the touchscreen 10. The pairing allows to highlight the instants tᵢ which correspond to the model instants tⱼ of the measurement made by the touchscreen 10 from those which do not correspond to any of the instants tⱼ.

Subsequent to this first sub-step 610), it is preferable to perform an evaluation 620) of the quality of the pairing made. The quality of the pairing is assessed in terms of the number of instants tᵢ that “overlap” or pair with the model instants tⱼ.

The quality of the pairing is evaluated by means of an objective function ƒ(δ) defined by:

ƒ(δ): =|{j∈[1,Nₜₐₚ]t.q. matches( [symbol] ,δ)=}| (12)

Where the function matches( [symbol] , δ) is defined by:

matches( [symbol] ,δ):=|{i,∈[1,ρNₜₐₚ]t.q. \tilde{t}+ρ−tᵢ<∈}| (13)

and ∈ is a threshold value (in milliseconds) that controls the maximum difference tolerated to consider the pairing as good.

Preferably, ∈ can range from 30 to 100 milliseconds.

Step 600) may further comprises a third sub-step 630) consisting in maximizing the objective function ƒ(δ) by means of a parameter δ^{opt}.

The parameter δ^{opt} is defined by:

δ^{opt}:=argmax_{δ∈[0,δ}_{ₘₐₓ}_{]}ƒ(δ) (14)

Where δₘₐₓ is an appropriate threshold value below \tilde{t}Nₜₐₚ.

At the end of the third sub-step 630), the quality of the pairing performed during the first sub-step 610) can be evaluated with a good accuracy and the pairing can be corrected if necessary.

A fourth selection sub-step 640) is then implemented. It consists in selecting the local maxima mₗᵢ associated to the sampling instants tᵢ which are exactly paired to the model instants tⱼ measured by the touchscreen.

At the end of this step, the signal [symbol] is basically cleaned of all unwanted peaks and the peaks corresponding to the vibro-acoustic events ₑᵥₐ are paired with the measurements of the touchscreen 10. In other words, the instants tᵢ of the maxima mₗᵢ correspond to the instants tⱼ of the measurements of the touchscreen 10.

Step 600) may comprises, subsequent to the fourth sub-step 640), a fifth adjustment sub-step 650) in which the local maxima mₗᵢ selected at the end of step 640) are adjusted to conform to the original signal, i.e. the recorded audio signal S_{E}. Indeed, the smoothing and filtering steps carried out during steps 300) and 400) may have introduced a phase shift in the signal that needs to be compensated.

This fourth adjustment step 640) is therefore performed by applying a phase shift compensation function to the maxima mₗᵢ= [symbol] (tᵢ). Said phase shift compensation function being defined by:

δ^{opt}:=argmax_{δ∈[0,δ}_{ₘₐₓ}_{]}ƒ(δ) (15)
Where tᵢ are the sampling instants associated with the local maxima mₗᵢ selected at the end of step 640),
   Tᵢ being the number of samples within the search time window, and
   [symbol] is the signal before smoothing.

At the end of the optimization step 600), the tap times of the user in response to the auditory stimulus, i.e. reference audio signal S_{R}, are determined with a very good accuracy.

In another embodiment of the present invention, it is possible to determine the tap times of the user without the user tapping on the touchscreen 10 in response to an auditory stimulus. In this embodiment, it would then not be necessary to perform steps of synchronizing the reference audio signal S_{R} with the recorded audio signal or even to perform the identification, i.e. the localization of the beginning, of said reference signal S_{R} in the recorded signal S_{E}.

1. A method for determining the tap times of a user in response to an auditory stimulus, the method comprising:
playing back the auditory stimulus, the auditory stimulus corresponding to a reference audio signal (S_{R});
recording the reference audio signal (S_{R}) and one or more vibro-acoustic events (eᵥₐ), the vibro-acoustic events (eᵥₐ) being subsequent to one or more taps by the user on a touchscreen of a touchscreen device in reaction to the reference audio signal (S_{R}), resulting in a recorded audio signal (S_{E}), wherein the recorded audio signal (S_{E}) is recorded with a microphone of the touchscreen device:
detecting the reference audio signal (S_{R}) in the recorded audio signal (S_{E})
the method further comprising
placing the reference audio signal (S_{R}) and the recorded audio signal (S_{E}) on a single common time scale;
filtering the recorded signal obtained subsequently and normalizing the recorded signal in such a way as to keep only the frequencies corresponding to the vibro-acoustic events (eᵥₐ) generated by the actions of the user on the touchscreen; and
detecting vibro-acoustic events (eᵥₐ) in which instants (tᵢ) associated with the vibro-acoustic events (eᵥₐ) are identified in the signal obtained.

2. The method according to claim 1, wherein detecting the reference audio signal further comprises normalizing the recorded audio signal (S_{E}) so as to obtain a normalized recorded audio signal (S_{E,n}).

3. The method according to claim 2, wherein detecting the reference audio signal further comprises resampling the reference audio signal (S_{R}) at the same rate as the recorded audio signal (S_{E}) and normalizing the reference audio signal (S_{R}) so as to obtain a resampled and normalized reference audio signal (S_{R,n}).

4. The method according to claim 2, wherein the normalization operation is performed with a normalization function defined by:
$${f}_{n} ( X ) := \frac{X - mean ( X )}{median ( abs ( X ) )}$$
Where X is the recorded audio signal (S_{E}) or the resampled reference audio signal (S_{R}), mean (X) is a mean value of the signal under consideration, abs (X) is a signal whose samples are absolute values of the samples of the signal X and median (abs(X)) is a median value of the signal abs (X).

5. The method according to claim 3, wherein detecting the reference audio signal further comprises, identifying the beginning of the resampled and normalized reference audio signal (S_{R,n}) in the normalized recorded audio signal (S_{E,n}).

6. The method according to claim 5, wherein detecting the reference audio signal further comprises:
constructing a suitable filter so as to identify in the normalized recorded audio signal (SE,n) a sampling instant (tSR) which corresponds to the beginning of K first samples of the resampled and normalized reference audio signal (SR,n);
determining a time window of size (TSR,n) in which to search for the normalized resampled reference audio signal (SR,n) in the normalized recorded audio signal (SE,n); and
determining the sampling instant (tSR).

7. The method according to claim 6, wherein the sampling instant (t_{SR}) is defined as follows:
$${t}_{SR} := {argmax}_{t < {T}_{SR , n}} {\sum }_{k = t}^{t + K} {S}_{E , n} ( k ) \cdot {S}_{R , n} ( k - t )$$
Where (t) corresponds to a given sampling instant, (t_{SR}) expresses the sampling instant corresponding to the beginning of the resampled and normalized reference audio signal (S_{R,n}) in the normalized recorded audio signal (S_{E,n}), (T_{SR,n}) is the size of the time window (in seconds), and argmax, denotes a set of points, over the time window (T_{SR,n}), at which the convolution product Σₖ₌ₜ^{t+K}S_{E,n} (k)·S_{R,n} (k−t) reaches its maximum value.

8. The method according to claim 1, wherein the filtering further comprises a first filtering step and a second filtering step.

9. The method according to claim 8, wherein:
the first filtering step is filtering with a 1st order Butterworth type bandpass filter having a low frequency of 50 Hz and a high frequency of 200 Hz; and
the second filtering step is filtering with a 1st order Butterworth type low-pass filter having a high frequency of 400 Hz,
obtaining a filtered normalized recorded audio signal (S_{E,n,f}).

10. The method according to claim 8, wherein the second filtering step further comprises locally normalizing a filtered normalized recorded audio signal (S_{E,n,f}) obtained so as to obtain a filtered normalized recorded audio signal ( [symbol] ), the local normalization defined by:
$${f}_{nloc} ( {S}_{E , n , f} ) := \frac{{S}_{E , n , f} - {mean}_{loc} ( {S}_{E , n , f} )}{{mean}_{loc} ( {abs}_{loc} ( {S}_{E , n , f} - {mean}_{loc} ( {S}_{E , n , f} ) ) )}$$
Where (S_{E,n,f}) is the filtered normalized recorded audio signal, mean_{loc} (S_{E,n,f}) is a local mean value of the filtered normalized recorded audio signal, and (S_{E,n,f}), abs_{loc} (S_{e,n,f}−mean_{loc} (S_{E,n,f})) is a signal whose the samples are local absolute values of the samples of the filtered normalized recorded audio signal (S_{E,n,f}) over the subset of a sliding window of size 2Tₙₒᵣₘ.

11. The method according to claim 8, further comprising detecting vibro-acoustic events (eᵥₐ).

12. The method according to claim 11, wherein detecting vibro-acoustic events comprises determining the energy ( [symbol] ) of a filtered normalized recorded audio signal ( [symbol] ) with an energy function defined by:
$$( t ) = {\sum }_{k = - {T}_{E}}^{{T}_{E}} {\vert ( t + k ) \vert}^{2}$$
Where (t) corresponds to a given sampling instant, (2T_{E}) is the size of a sliding time window, the number of samples being equal to 2T_{E} in the sliding window, and ( [symbol] ) is the filtered normalized recorded audio signal obtained.

13. The method according to claim 12, wherein detecting vibro-acoustic events further comprises smoothing the signal ( [symbol] ) obtained at the end of the first sub step with a smoothing function defined by the convolution product of the signal ( [symbol] ) with a Hamming-type weighting window: [symbol]
:= [symbol] *Hamming(T_{lissage})
Where ( [symbol] )is the smoothed signal, ( [symbol] ) is the signal before smoothing, Hamming is the weighting window, and (T_{smoothing}) is the size of the weighting window.

14. The method according to claim 13, wherein detecting vibro-acoustic events further comprises extracting from the smoothed signal ( [symbol] ), a set of P sampling instants (tᵢ) corresponding to local maxima (mli) and/or onset candidates of vibro-acoustic events (eᵥₐ), the sampling instants being such that [symbol]
(tᵢ)> [symbol] (tᵢ₋₁) and [symbol]
(tᵢ)> [symbol] (tᵢ₊₁)

15. The method according to claim 14, wherein detecting vibro-acoustic events further comprises pre-selecting the onset candidates of vibro-acoustic event (eᵥₐ) by:
grouping candidates (mₗᵢ) associated with the P sampling instants (tᵢ) according to a first selection criterion, the first selection criterion corresponding to the grouping of candidate sampling instants spaced by a predetermined number m of samples, so as to form groups (gⱼ) of local maxima (mₗᵢ); and
conserving in each group gⱼ the instants associated with the local maxima (mₗᵢ) for which the maximum value of [symbol] is obtained.

16. The method according to claim 15, wherein detecting vibro-acoustic events further comprises removing the spurious maxima (mₗᵢ) by:
sorting, by decreasing height, the local maxima (mₗᵢ); and
conserving a ρNₜₐₚ largest local maxima (mₗᵢ), Nₜₐₚ being the number of vibro-acoustic events (eᵥₐ) comprised in the measurement time window and ρ being strictly greater than 1, ρ>1.

17. The method according to claim 16, further comprising, optimizing the signal obtained by:
pairing the set of the instants (tᵢ), with i<ρNₜₐₚ, of the local maxima (mₗᵢ) with a set of model instants (tⱼ), with j<Nₜₐₚ, measured by the touchscreen,
evaluating the quality of the pairing with an objective function (ƒ(δ)),
maximizing the objective function (ƒ(δ)) with a parameter (δ^{opt}),
selecting the local maxima (mₗᵢ) associated with the sampling instants (tᵢ) which are exactly paired to the model instants (tⱼ) measured by the touchscreen.

18. The method according to claim 17, wherein the objective function is defined by:
ƒ(δ): =|{j∈[1,Nₜₐₚ]t.q. matches( [symbol] ,δ)=1}|

Where the function matches( [symbol] , δ) is defined by:
matches( [symbol] ,δ):=|{i∈[1,ρNₜₐₚ]t.q. \tilde{t}+ρ−tᵢ<∈}|

and (∈) is a threshold value (in milliseconds) that controls the maximum difference tolerated to consider the pairing is of quality.

19. The method according to claim 17, wherein the parameter (δ^{opt}) is defined by:
δ^{opt}:=argmax_{δ∈[0,δ}_{ₘₐₓ}_{]}ƒ(δ)
Where δₘₐₓ is an appropriate threshold value below \tilde{t} Nₜₐₚ.

20. The method according to claim 17, wherein optimizing the signal obtained further comprises adjusting the local maxima (mₗᵢ) selected so as to conform to the recorded audio signal (S_{E}).

21. The method according to claim 20, wherein the adjustment is performed by applying a phase shift compensation function to the maxima mₗᵢ= [symbol] (tᵢ), the function being defined by: [symbol]
:=argmax_{t∈[t}_{ᵢ}_{-T}_{ᵢ}_{,t}_{ᵢ}_{+T}_{ᵢ}_{]} [symbol] (tᵢ)
Where (tᵢ) is the sampling instant associated with the local maxima mli selected, (Tᵢ) being a number of samples comprised in the search time window, ( [symbol] ) is the signal before smoothing.

22. A touchscreen device suitable for carrying out the method according to claim 1, the device comprising:
a touchscreen;
a microphone; and
a central processing unit configured to perform at least steps of the method according to claim 1.

23. A computer program comprising instructions which when executed by the computer cause the computer to carry out the method of claim 1.

24. A non-transitory computer readable medium comprising instructions stored thereon, which when executed by one or more processor circuits causes the one or more processor circuits to carry out the method of claim 1.
